(19) United States
(12) Patent Application Publication
(10) Pub. No.: US 2010/0000032 A1
(43) Pub. Date: Jan. 7, 2010

(54) PHENOXAZINE DYES

(75) Inventors: Frederic Bruyneel, Mont-Saint-Guibert (BE); Riccardo Basosi, Firenze (IT); Christian-Marie Bols, Chastre (BE); Estelle Enaud, Mont-Saint-Guibert (BE); Christoph Hercher, Mullheim (DE); Jutta Ismene Jager, Umkirch (DE); Anna Jarosz-Wilkolazka, Swidnik (PL); Jacqueline Marchand-Brynaert, Marcinelle (BE); Rebecca Pogni, Siena (IT); Jolanta Polak, Lublin (PL); Sophie Vanhulle, Chastre (BE)

Correspondence Address: KNOBBE MARTENS OLSON & BEAR LLP, 2040 MAIN STREET, FOURTEENTH FLOOR, IRVINE, CA, 92614, US

(73) Assignees: Wetlands Incubator sprl, Louvain-la-Neuve (BE); Setas Kimya San AS, Istanbul (TR)

(21) Appl. No.: 12/498,666

(22) Filed: Jul. 7, 2009

Related U.S. Application Data
(60) Provisional application No. 61/078,670, filed on Jul. 7, 2008.

(51) Int. Cl. C09B 19/00; C07D 265/38; C12P 17/14

(52) U.S. Cl. 8/657; 544/104; 544/102; 435/120

(57) ABSTRACT

The present invention concerns highly water-soluble phenoxazines dyes of formula (I)

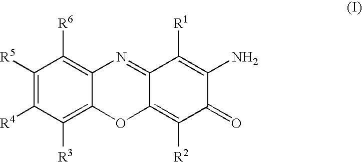

wherein R¹, R², R³, R⁴, R⁵ and R⁶ have the meaning defined in the claim, with improved photochemical properties, low toxicity, and favourable solubility in water. It also concerns a method for their preparation.

### RELATED APPLICATIONS

This application claims priority to U.S. Provisional Application No. 61/078,670, filed Jul. 7, 2008, which is incorporated herein by reference in its entirety.

### FIELD OF THE INVENTION

The present invention concerns highly water-soluble phenoxazines dyes with improved photochemical properties and solubility in water.

### BACKGROUND TO THE INVENTION

Cinnabarinic acid is a red phenoxazinone compound responsible for the Pycnoporus species colour. Phenoxazinones have been identified in various biological systems including pigments and antibiotics. For instance, actinomycin is an antibiotic which is formed by phenoxazinone synthase, an enzyme of Streptomyces species. The search of water-soluble long-wavelength fluorochromophores for the detection of biological and organic molecules has recently drawn high interest. Fluorescence-based techniques are sensitive, simple and selective, making them ideal for screening techniques, particularly using arrays. However, the poor solubility of the currently available phenoxazinone derivatives represents a drawback for biological applications.

It is an aim of the present invention to provide highly water-soluble phenoxazinone dyes that have improved photochemical properties useful for industrial and domestic applications.

Synthesis of phenoxazinone dyes by substitution to the phenoxazinone core is difficult to control. Reactions for the synthesis of sophisticated analogs of 3-H-phenoxazin-3-one by halogenation have so far failed. Furthermore, the usual chemical synthesis of phenoxazine derivatives involves the condensation of nitroso compounds at elevated temperature. Such processes are highly toxic and energy consuming. Moreover, the cyclization of sulfonated nitrosoanilines as building blocks is not very effective. It is an aim to provide a methodology for producing new amino-phenoxazinones, that overcomes at least one of the problems of the art.

### SUMMARY OF THE INVENTION

One embodiment of the invention is a compound of structural formula (I), a tautomer, a quaternary form or salt thereof,

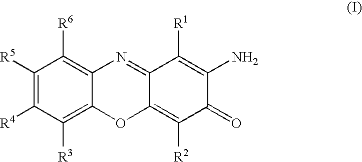

wherein
R¹ is selected from a group consisting of H, —SO₃H, —SO₃M, —SO₂NR⁷R⁸, —SO₂H, —SO₂—C₁₋₆alkyl, —SO₂—C₆₋₁₂aryl, —SO₂O—C₁₋₆alkyl, and —SO₂O—C₆₋₁₂aryl;
R² is selected from the group consisting of H, —SO₃H, —SO₃M, —SO₂NR⁹R¹⁰, —SO₂H, —SO₂—C₁₋₆alkyl, —SO₂—C₆₋₁₂aryl, —SO₂O—C₁₋₆alkyl, —SO₂O—C₆₋₁₂aryl, C₁₋₆alkyl, C₆₋₁₂aryl, SH, OH, —NH₂, halogen, carboxyl, aminocarbonyl, C₁₋₆alkylaminocarbonyl, and C₆₋₁₂arylaminocarbonyl;
R³ is selected from the group consisting of H, —SO₃H, —SO₃M, —SO₂NR¹¹R¹²—SO₂H, —SO₂—C₁₋₆alkyl, —SO₂—C₆₋₁₂aryl, —SO₂O—C₁₋₆alkyl, —SO₂O—C₆₋₁₂aryl, C₁₋₆alkyl, C₆₋₁₂aryl, SH, OH, —NH₂, halogen, carboxyl, aminocarbonyl, C₁₋₆alkylaminocarbonyl, and C₆₋₁₂arylaminocarbonyl;
R⁴ is selected from the group consisting of H, —SO₃H, —SO₃M, —SO₂NR¹³R¹⁴, —SO₂H, —SO₂—C₁₋₆alkyl, —SO₂—C₆₋₁₂aryl, —SO₂O—C₁₋₆alkyl, —SO₂O—C₆₋₁₂aryl, halogen, aminocarbonyl, C₁₋₆alkylaminocarbonyl, C₆₋₁₂arylaminocarbonyl, C₁₋₆alkyl, C₆₋₁₂aryl, SH, OH, and —NH₂;
R⁵ is selected from the group consisting of H, —SO₃H, —SO₃M, —SO₂NR¹⁵R¹⁶, —SO₂H, —SO₂—C₁₋₆alkyl, —SO₂—C₆₋₁₂aryl, —SO₂O—C₁₋₆alkyl, —SO₂O—C₆₋₁₂aryl, halogen, aminocarbonyl, C₁₋₆alkylaminocarbonyl, C₆₋₁₂arylaminocarbonyl, C₁₋₆alkyl, C₆₋₁₂aryl, SH, OH, and —NH₂;
R⁶ is selected from the group consisting of H, —SO₃H, —SO₃M, —SO₂NR¹⁷R¹⁸, —SO₂H, —SO₂—C₁₋₆alkyl, —SO₂—C₆₋₁₂aryl, —SO₂O—C₁₋₆alkyl, and —SO₂O—C₆₋₁₂aryl;
wherein at least one of R¹, R², R³, R⁴, R⁵ and R⁶ is one of the —SO₂— or —SO₃— containing moieties listed above,
wherein
R⁷ and R⁸ are each independently selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, and C₆₋₁₂aryl;
R⁹ and R¹⁰ are each independently selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, and C₆₋₁₂aryl;
R¹¹ and R¹² are each independently selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, and C₆₋₁₂aryl;
R¹³ and R¹⁴ are each independently selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, and C₆₋₁₂aryl;
R¹⁵ and R¹⁶ are each independently selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, and C₆₋₁₂aryl;
R¹⁷ and R¹⁸ are each independently selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, and C₆₋₁₂aryl, and
M is selected from the group consisting of Li, Na, K, Cs, and Rb.

Another embodiment of the invention is a compound as described above, wherein R² is hydrogen and R¹, R³, R⁴, R⁵, R⁶ have the meaning as defined above.

Another embodiment of the invention is a compound as described above, wherein R³ is hydrogen and R¹, R², R⁵ and R⁶ have the meaning as defined above.

Another embodiment of the invention is a compound as described above, wherein R² and R³ are each independently hydrogen and R¹, R⁴, R⁵ and R⁶ have the meaning as defined above.

Another embodiment of the invention is a compound as described above, having the structural formula (II)

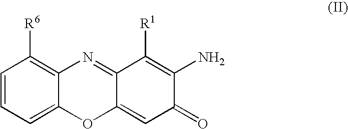

wherein R¹ and R⁶ have the meaning defined above.

Another embodiment of the invention is a compound as described above, having structural formula (II), wherein R¹ is H, and R⁶ is has the meaning defined above.

Another embodiment of the invention is a compound as described above, having structural formula (II) wherein R¹ is has the meaning defined above, and R⁶ is H.

Another embodiment of the invention is a compound as described above, having structural formula (II), wherein

R¹ is selected from a group consisting of —SO₃H, —SO₃M, —SO₂NR⁷R⁸, —SO₂—C₁₋₆alkyl, —SO₂—C₆₋₁₂aryl, —SO₂O—C₁₋₆alkyl, and —SO₂O—C₆₋₁₂aryl,
R⁶ is selected from the group consisting of —SO₃H, —SO₃M, —SO₂NR¹⁷R¹⁸, —SO₂—C₁₋₆alkyl, —SO₂—C₆₋₁₂aryl, —SO₂O—C₁₋₆alkyl, and —SO₂O—C₆₋₁₂aryl, and
R¹⁷ and R¹⁸ have the meaning as defined above.

Another embodiment of the invention is a compound as described above, having the structural formula (III)

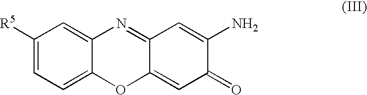

wherein R⁵ has the meaning defined above.

Another embodiment of the invention is a compound as described above, having the structural formula (III), wherein R⁵ is selected from a group consisting of —SO₃H, —SO₃M, —SO₂NR¹⁵R¹⁶, —SO₂—C₁₋₆alkyl, —SO₂—C₆₋₁₂aryl, —SO₂O—C₁₋₆alkyl, and —SO₂O—C₆₋₁₂aryl, and R¹⁵ and R¹⁶ have the meaning as defined above.

Another embodiment of the invention is a compound as described above, that is a compound selected from the group consisting of 2-amino-3-oxo-3H-phenoxazine-1,9-disulfonic acid diamide, 2-amino-3-oxo-3H-phenoxazine-1,9-disulfonic acid bis-phenylamide, 2-amino-3-oxo-3H-phenoxazine-1,9-disulfonic acid bis-cyclohexylamide, 2-amino-3-oxo-3H-phenoxazine-1,9-disulfonic acid bis-[(3-dimethylamino-propyl)-amide, 2-amino-3-oxo-3H-phenoxazine-1,9-disulfonic acid bis-[(2-amino-ethyl)-amide], 2-amino-[9-(methoxycarbonylmethyl-sulfamoyl)-7-oxo-7H-phenoxazine-1-sulfonylamino]-acetic acid methyl ester, 2-amino-3-oxo-3H-phenoxazine-1,9-disulfonic acid bis-[(3-hydroxy-propyl)amide].

Another embodiment of the invention is a method for the preparation of a compound of formula (I) as defined above, comprising the step of catalytically dimerising an aromatic precursor having structural formula (IV),

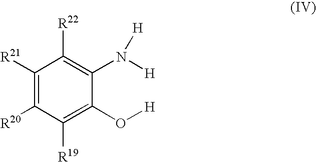

wherein R¹⁹ is the same as R² or R³ as defined above, R²⁰ is the same as R⁴ as defined above, R²¹ is the same as R⁵ as defined above, and R²² is the same as R¹ or R⁶ as defined above, whereby the catalysis proceeds using a laccase enzyme.

Another embodiment of the invention is a method as described above, wherein the laccase enzyme is in free solution or immobilised on a solid support, or is provided by a micro-organism containing a gene for laccase enzyme expression.

Another embodiment of the invention is a method as described above, wherein the reaction proceeds in an aqueous medium.

Another embodiment of the invention is a method as described above, wherein the medium further comprises an alcohol at a concentration of between 1 and 10% (v/v).

Another embodiment of the invention is a method as described above, wherein the laccase enzyme is present at a concentration that provides an activity of between 1 and 1000 U·L⁻¹.

Another embodiment of the invention is a method as described above, wherein the compound of formula (IV) is present at a concentration of between 1 and 10 g·L⁻¹.

Another embodiment of the invention is a method of dyeing an article comprising the step of contacting said article with a compound as defined above.

Another embodiment of the invention is an article dyed using a compound as defined above.

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

### DETAILED DESCRIPTION OF THE INVENTION

The present invention relates to a synthetic dye compound that is a sulfonated phenoxazinone derivative, and which finds application, for example, in colouring textiles and dyeing of hair. The present invention also concerns a bioprocess for the synthesis of sulfonated phenoxazinone derivatives, including the dye compounds of the present invention, using the enzyme laccase. The use of enzymes integrated with traditional synthetic methods provides a more environmentally friendly process, avoiding the use of large quantities of organic solvents and other potentially toxic reagents.

The dyes of the present invention are highly water-soluble compared with phenoxazines dyes known in the art such as Meldola Blue and Nile Red. Cinnabarinic acid, Meldola Blue or Nile Red dyes have a poor solubility in water making them unsuitable for a large number of applications. In addition, the dyes of the present invention have improved photochemical properties, exhibiting greater stability on exposure to natural light i.e. dyed articles retain good colour saturation over time, and dyed textiles show favourable colour fastness. Furthermore, the new phenoxazinone derivatives are non-toxic, and offer strong colour saturation, for instance, a high molar extinction coefficient (e.g. around 450 nm). These novel phenoxazinones have utility as industrial dyes for textile, or for domestic or professional use in hair dyeing applications.

As used in the foregoing and hereinafter, the following definitions apply unless otherwise noted.

As used herein “C₁₋₆alkyl”, as a group or part of a group, defines straight or branched chain saturated hydrocarbon radicals having from 1 to 6 carbon atoms such as for example methyl, ethyl, prop-1-yl, prop-2-yl, but-1-yl, but-2-yl, isobutyl, 2-methyl-prop-1-yl; pent-1-yl, pent-2-yl, pent-3-yl, hex-1-yl, hex-2-yl, 2-methylbut-1-yl, 2-methylpent-1-yl, 2-ethylbut-1-yl, 3-methylpent-2-yl, and the like. Of interest amongst C₁₋₆alkyl is C₁₋₄alkyl.

Where C₁₋₆alkyl groups as defined are divalent, i.e., with two single bonds for attachment to two other groups, they are termed “C₁₋₆alkylene” groups. Non-limiting examples of alkylene groups includes methylene, ethylene, methylmethylene, propylene, ethylethylene, 1,2-dimethylethylene, and the like.

The term “C₄₋₆cycloalkyl”, as a group or part of a group, is a cyclic alkyl group, that is to say, a monovalent, saturated hydrocarbyl group comprising from 4 to 6 carbon atoms and having 1 cyclic structure. Examples of C₄₋₆ cycloalkyl groups include but are not limited to cyclopropyl, cyclobutyl, cyclopentyl, cyclohexyl. When the suffix “ene” is used in conjunction with a cyclic group, this is intended to mean the cyclic group as defined herein having two single bonds as points of attachment to other groups.

The term “C₆₋₁₂aryl”, as a group or part of a group, refers to a polyunsaturated, aromatic hydrocarbyl group having a single ring (i.e. phenyl) or multiple aromatic rings fused together (e.g. naphthalene), or linked covalently, typically containing 6 to 12 atoms; wherein at least one ring is aromatic. Non-limiting examples of C₆₋₁₂aryl comprise phenyl, biphenylyl, biphenylenyl, or 1- or 2-naphthanelyl.

Where C₆₋₁₂aryl groups as defined are divalent, i.e., with two single bonds for attachment to two other groups, they are termed “C₁₋₆arylene” groups. Non-limiting examples of arylene groups includes phenylene and the like.

The term “carboxy” or “carboxyl”, as a group or part of a group, refers to the group —CO₂H.

The term “aminocarbonyl”, as a group or part of a group, refers to the group —(C═O)—NH₂.

The term “C₁₋₆alkylaminocarbonyl”, as a group or part of a group, refers to a group —(C═O)—NR^{a}R^{b} wherein R^{a} is hydrogen or C₁₋₆alkyl as defined above, and R^{b} is C₁₋₆alkyl as defined above.

The term “C₆₋₁₂arylaminocarbonyl”, as a group or part of a group, refers to a group of formula —C(═O)—N(R^{c})(R^{d}) wherein R^{c} is hydrogen, C₆₋₁₂aryl or C₁₋₆alkyl as defined above, and R^{d} is C₆₋₁₂aryl as defined above.

The term halo or halogen is generic to fluoro, chloro, bromo and iodo.

The term “amino”, as a group or part of a group, refers to the group —NH₂.

The term “aminoC₁₋₆alkyl”, as a group or part of a group, refers to the group —R^{e}—NR^{a}R^{f} wherein R^{e} is C₁₋₆alkylene, R^{a} is hydrogen or C₁₋₆alkyl as defined herein, and R^{f} is hydrogen or C₁₋₆alkyl as defined herein.

The term “C₁₋₆alkylaminoC₁₋₆alkyl”, as a group or part of a group, refers to a group of formula —R^{e}—N(R^{a})(R^{b}) wherein R^{e} is C₁₋₆alkylene as defined above, R^{a} is hydrogen or C₁₋₆alkyl as defined above, and R^{b} is C₁₋₆alkyl as defined above.

The term “C₁₋₆alkoxycarbonylC₁₋₆alkyl” or “C₁₋₆alkyloxycarbonylC₁₋₆alkyl”, as a group or part of a group, refers to a radical having the Formula —R^{e}—C(═O)OR^{b} wherein R^{e} is a C₁₋₆alkylene, and R^{b} is C₁₋₆alkyl as defined above.

The term “hydroxyC₁₋₆alkyl”, as a group or part of a group, refers to the group —R^{e}—OH wherein R^{e} is C₁₋₆alkylene.

It should be noted that the group positions on any molecular moiety used in the definitions may be anywhere on such moiety as long as it is chemically stable.

Groups used in the definitions of the variables include all possible isomers unless otherwise indicated. For instance naphthalenyl includes naphthalen-1-yl and naphthalen-2-yl.

When any variable occurs more than one time in any constituent, each definition is independent.

As used in the specification and the appended claims, the singular forms “a”, “an”, and “the” include plural referents unless the context clearly dictates otherwise. By way of example, “a compound” means one compound or more than one compound.

Some of the compounds of formula (I) may exist in their tautomeric form. Such forms although not explicitly indicated in the above formula are intended to be included within the scope of the present invention.

The term “salt thereof” as used encompasses the fact that compounds of Formula (I) contain an acidic proton which can also be converted into their non-toxic metal or amine addition salt forms by treatment with appropriate organic and inorganic bases. Appropriate base salt forms comprise, for example, the ammonium salts, the alkali and earth alkaline metal salts, e.g. the lithium, sodium, potassium, magnesium, calcium salts and the like, salts with organic bases, e.g. primary, secondary and tertiary aliphatic and aromatic amines such as methylamine, ethylamine, propylamine, isopropylamine, the four butylamine isomers, dimethylamine, diethylamine, diethanolamine, dipropylamine, diisopropylamine, di-n-butylamine, pyrrolidine, piperidine, morpholine, trimethylamine, triethylamine, tripropylamine, quinuclidine, pyridine, quinoline and isoquinoline; the benzathine, N-methyl-D-glucamine, hydrabamine salts, and salts with amino acids such as, for example, arginine, lysine and the like. Conversely the salt form can be converted by treatment with acid into the free acid form.

The term “quaternary form” as used hereinbefore defines the quaternary ammonium salts which the compounds of Formula (I) are able to form by reaction between a basic nitrogen of a compound of Formula (I) and an appropriate quaternizing agent, such as, for example, an optionally substituted alkylhalide, arylhalide or arylalkylhalide, e.g. methyliodide or benzyliodide. Other reactants with good leaving groups may also be used, such as alkyl trifluoromethanesulfonates, alkyl methanesulfonates, and alkyl p-toluenesulfonates. A quaternary amine has a positively charged nitrogen.

One embodiment of the invention is a compound of the formula (I) wherein

R¹ is selected from a group consisting of H, —SO₃H, —SO₃M, —SO₂NR⁷R⁸, —SO₂H, —SO₂—C₁₋₆alkyl, —SO₂—C₆₋₁₂aryl, —SO₂O—C₁₋₆alkyl, and —SO₂O—C₆₋₁₂aryl; preferably R¹ is selected from a group consisting of H, —SO₃H, —SO₃M, —SO₂NR⁷R⁸, —SO₂—C₁₋₆alkyl, —SO₂—C₆₋₁₀aryl, —SO₂O—C₁₋₆alkyl, and —SO₂O—C₆₋₁₀aryl; preferably R¹ is selected from a group consisting of H, —SO₃H, —SO₃M, —SO₂NR⁷R⁸, —SO₂—C₁₋₄alkyl, —SO₂—C₆₋₁₀aryl; preferably R¹ is selected from a group consisting of H, —SO₃H, —SO₃M, —SO₂NR⁷R⁸; preferably R¹ is H; preferably R¹ is —SO₃H, preferably R¹ is —SO₃M, preferably R¹ is —SO₂NR⁷R⁸;
R² is selected from the group consisting of H, —SO₃H, —SO₃M, —SO₂NR⁹R¹⁰, —SO₂H, —SO₂—C₁₋₆alkyl, —SO₂—C₆₋₁₂aryl, —SO₂O—C₁₋₆alkyl, —SO₂O—C₆₋₁₂aryl, C₁₋₆alkyl, C₆₋₁₂aryl, SH, OH, —NH₂, halogen, carboxyl, aminocarbonyl, C₁₋₆alkylaminocarbonyl, and C₆₋₁₂arylaminocarbonyl; preferably R² is selected from the group consisting of H, —SO₃H, —SO₃M, —SO₂NR⁹R¹⁰, —SO₂H, —SO₂—C₁₋₆alkyl, —SO₂—C₆₋₁₂aryl, —SO₂O—C₁₋₆alkyl, —SO₂O—C₆₋₁₂aryl, C₁₋₆alkyl, C₆₋₁₂aryl, OH, —NH₂, halogen, carboxyl, aminocarbonyl; preferably R² is selected from the group consisting of H, —SO₃H, —SO₃M, —SO₂NR⁹R¹⁰, C₁₋₆alkyl, C₆₋₁₂aryl, OH, —NH₂, halogen, carboxyl, aminocarbonyl; preferably R² is selected from the group consisting of H, —SO₃H, —SO₃M, —SO₂NR⁹R¹⁰, C₁₋₄alkyl, C₆₋₁₀aryl, OH, —NH₂, halogen, carboxyl, aminocarbonyl; preferably R² is selected from the group consisting of H, —SO₃H, —SO₃M, —SO₂NR⁹R¹⁰, phenyl, methyl, ethyl, propyl, isopropyl, butyl, isobutyl, tert-butyl, OH, —NH₂, halogen, carboxyl, aminocarbonyl; preferably R² is selected from the group consisting of H, —SO₃H, —SO₃M; preferably R² is H;
R³ is selected from the group consisting of H, —SO₃H, —SO₃M, —SO₂NR⁹R¹⁰, —SO₂H, —SO₂—C₁₋₆alkyl, —SO₂—C₆₋₁₂aryl, —SO₂O—C₁₋₆alkyl, —SO₂O—C₆₋₁₂aryl, C₁₋₆alkyl, C₆₋₁₂aryl, SH, OH, —NH₂, halogen, carboxyl, aminocarbonyl, C₁₋₆alkylaminocarbonyl, and C₆₋₁₂arylaminocarbonyl; preferably R³ is selected from the group consisting of H, —SO₃H, —SO₃M, —SO₂NR⁹R¹⁰, —SO₂H, —SO₂—C₁₋₆alkyl, —SO₂—C₆₋₁₂aryl, —SO₂O—C₁₋₆alkyl, —SO₂O—C₆₋₁₂aryl, C₁₋₆alkyl, C₆₋₁₂aryl, OH, —NH₂, halogen, carboxyl, aminocarbonyl; preferably R³ is selected from the group consisting of H, —SO₃H, —SO₃M, —SO₂NR⁹R¹⁰, C₁₋₆alkyl, C₆₋₁₂aryl, OH, —NH₂, halogen, carboxyl, aminocarbonyl; preferably R³ is selected from the group consisting of H, —SO₃H, —SO₃M, —SO₂NR⁹R¹⁰, C₁₋₄alkyl, C₆₋₁₀aryl, OH, —NH₂, halogen, carboxyl, aminocarbonyl; preferably R³ is selected from the group consisting of H, —SO₃H, —SO₃M, —SO₂NR⁹R¹⁰, phenyl, methyl, ethyl, propyl, isopropyl, butyl, isobutyl, tert-butyl, OH, —NH₂, halogen, carboxyl, aminocarbonyl; preferably R³ is selected from the group consisting of H, —SO₃H, —SO₃M; preferably R³ is H;
R⁴ is selected from the group consisting of H, —SO₃H, —SO₃M, —SO₂NR¹³R¹⁴, —SO₂H, —SO₂—C₁₋₆alkyl, —SO₂—C₆₋₁₂aryl, —SO₂O—C₁₋₆alkyl, —SO₂O—C₆₋₁₂aryl, halogen, aminocarbonyl, C₁₋₆alkylaminocarbonyl, C₆₋₁₂arylaminocarbonyl, C₁₋₆alkyl, C₆₋₁₂aryl, SH, OH, and —NH₂; preferably R⁴ is selected from the group consisting of H, —SO₃H, —SO₃M, —SO₂NR¹³R¹⁴, —SO₂O—C₁₋₆alkyl, —SO₂O—C₆₋₁₂aryl, halogen, aminocarbonyl, C₁₋₆alkylaminocarbonyl, C₁₋₆alkyl, C₆₋₁₂aryl, SH, OH, —NH₂; preferably R⁴ is selected from the group consisting of H, —SO₃H, —SO₃M, —SO₂NR¹³R¹⁴, halogen, C₁₋₆alkyl, C₆₋₁₂aryl, OH, —NH₂; preferably R⁴ is selected from the group consisting of H, —SO₃H, —SO₃M, —SO₂NR¹³R¹⁴, halogen, C₁₋₄alkyl, C₆₋₁₀aryl, OH, —NH₂; preferably R⁴ is selected from the group consisting of H, —SO₃H, —SO₃M, —SO₂NR¹³R¹⁴, halogen, methyl, ethyl, propyl, isopropyl, butyl, isobutyl, tert-butyl, phenyl, OH, —NH₂; preferably R⁴ is selected from the group consisting of H, —SO₃H, —SO₃M, halogen, methyl, ethyl, propyl, isopropyl, butyl, isobutyl, tert-butyl; preferably R⁴ is selected from the group consisting of H, halogen, methyl, ethyl, propyl, isopropyl, butyl, isobutyl, tert-butyl; preferably R⁴ is H;
R⁵ is selected from the group consisting of H, —SO₃H, —SO₃M, —SO₂NR¹⁵R¹⁶, —SO₂H, —SO₂—C₁₋₆alkyl, —SO₂—C₆₋₁₂aryl, —SO₂O—C₁₋₆alkyl, —SO₂O—C₆₋₁₂aryl, halogen, aminocarbonyl, C₁₋₆alkylaminocarbonyl, C₆₋₁₂arylaminocarbonyl, C₁₋₆alkyl, C₆₋₁₂aryl, SH, OH, and —NH₂; preferably R⁵ is selected from the group consisting of H, —SO₃H, —SO₃M, —SO₂NR¹⁵R¹⁶, —SO₂O—C₁₋₆alkyl, —SO₂O—C₆₋₁₂aryl, halogen, aminocarbonyl, C₁₋₆alkylaminocarbonyl, C₆₋₁₂arylaminocarbonyl, C₁₋₆alkyl, C₆₋₁₂aryl, SH, OH, —NH₂; preferably R⁵ is selected from the group consisting of H, —SO₃H, —SO₃M, —SO₂NR¹⁵R¹⁶, halogen, aminocarbonyl, C₁₋₆alkylaminocarbonyl, C₁₋₆alkyl, C₆₋₁₂aryl, SH, OH, —NH₂; preferably R⁵ is selected from the group consisting of H, —SO₃H, —SO₃M, —SO₂NR¹⁵R¹⁶, halogen, C₁₋₆alkyl, C₆₋₁₂aryl, SH, OH, —NH₂; preferably R⁵ is selected from the group consisting of H, —SO₃H, —SO₃M, —SO₂NR¹⁵R¹⁶, halogen, C₁₋₄alkyl, C₆₋₁₀aryl, SH, OH, —NH₂; preferably R⁵ is selected from the group consisting of H, —SO₃H, —SO₃M, —SO₂NR¹⁵R¹⁶, halogen, methyl, ethyl, propyl, isopropyl, butyl, isobutyl, tert-butyl, phenyl, SH, OH, —NH₂; preferably R⁵ is selected from the group consisting of H, —SO₃H, —SO₃M, —SO₂NR¹⁵R¹⁶, OH, —NH₂; preferably R⁵ is H, —SO₃H, or —SO₃M; preferably R⁵ is H, preferably R⁵ is —SO₃H or —SO₃M;
R⁶ is selected from a group consisting of H, —SO₃H, —SO₃M, —SO₂NR⁷R⁸, —SO₂H, —SO₂—C₁₋₆alkyl, —SO₂—C₆₋₁₂aryl, —SO₂O—C₁₋₆alkyl, and —SO₂O—C₆₋₁₂aryl; preferably R⁶ is selected from a group consisting of H, —SO₃H, —SO₃M, —SO₂NR⁷R⁸, —SO₂—C₁₋₆alkyl, —SO₂—C₆₋₁₀aryl, —SO₂O—C₁₋₆alkyl, and —SO₂O—C₆₋₁₀aryl; preferably R⁶ is selected from a group consisting of H, —SO₃H, —SO₃M, —SO₂NR⁷R⁸, —SO₂—C₁₋₄alkyl, —SO₂—C₆₋₁₀aryl; preferably R⁶ is selected from a group consisting of H, —SO₃H, —SO₃M, —SO₂NR⁷R⁸; preferably R⁶ is H; preferably R⁶ is —SO₃H, preferably R⁶ is —SO₃M, preferably R⁶ is —SO₂NR⁷R⁸;
wherein at least one of R¹, R², R³, R⁴, R⁵ and R⁶ is one of the —SO₂— or —SO₃— containing moieties listed above, preferably wherein at least one of R¹, R⁴, R⁵ and R⁶ is one of the —SO₂— or —SO₃— containing moieties listed above, preferably wherein at least one of R¹, R⁵ and R⁶ is one of the —SO₂— or —SO₃— containing moieties listed above,
wherein
R⁷ and R⁸ are each independently selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, and C₆₋₁₂aryl; preferably R⁸ is hydrogen or C₁₋₆alkyl, and R⁷ is selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, C₆₋₁₂aryl; preferably R⁷ and R⁸ are each independently selected from the group consisting of H, C₁₋₆alkyl, C₁₋₄alkylaminoC₁₋₆alkyl, aminoC₁₋₆alkyl, cyclopropyl, cyclobutyl, cyclopentyl, cyclohexyl, C₁₋₄alkoxycarbonylC₁₋₆alkyl, hydroxyC₁₋₄alkyl, phenyl, naphthalenyl; preferably R⁸ is hydrogen or C₁₋₄alkyl, and R⁷ is selected from the group consisting of H, C₁₋₄alkyl, C₁₋₄alkylaminoC₁₋₄alkyl, aminoC₁₋₄alkyl, cyclopropyl, cyclobutyl, cyclopentyl, cyclohexyl, C₁₋₄alkoxycarbonylC₁₋₄alkyl, hydroxyC₁₋₄alkyl, phenyl; preferably R⁸ is hydrogen, and R⁷ is selected from the group consisting of H, methyl, ethyl, propyl, isopropyl, butyl, isobutyl, tert-butyl, C₁₋₄alkylaminoC₁₋₄alkyl, aminoC₁₋₄alkyl, cyclopropyl, cyclobutyl, cyclopentyl, cyclohexyl, C₁₋₄alkoxycarbonylC₁₋₄alkyl, hydroxymethyl, hydroxyethyl, hydroxypropyl, hydroxybutyl, phenyl; preferably R³ is hydrogen, and R⁷ is selected from the group consisting of H, methyl, ethyl, propyl, isopropyl, butyl, isobutyl, tert-butyl, C₁₋₄alkylaminomethyl, C₁₋₄alkylaminoethyl, C₁₋₄alkylaminopropyl, C₁₋₄alkylaminobutyl, aminomethyl, aminoethyl, aminopropyl, aminobutyl, cyclopropyl, cyclobutyl, cyclopentyl, cyclohexyl, C₁₋₄alkoxycarbonylmethyl, C₁₋₄alkoxycarbonylethyl, C₁₋₄alkoxycarbonylbutyl, C₁₋₄alkoxycarbonylpropyl, hydroxymethyl, hydroxyethyl, hydroxypropyl, hydroxybutyl, phenyl; preferably R⁸ is hydrogen, and R⁷ is selected from the group consisting of H, methyl, ethyl, propyl, isopropyl, butyl, isobutyl, tert-butyl, C₁₋₄alkylaminomethyl, C₁₋₄alkylaminoethyl, C₁₋₄alkylaminopropyl, C₁₋₄alkylaminobutyl, aminomethyl, aminoethyl, aminopropyl, aminobutyl, cyclopentyl, cyclohexyl, C₁₋₄alkoxycarbonylmethyl, C₁₋₄alkoxycarbonylethyl, C₁₋₄alkoxycarbonylbutyl, C₁₋₄alkoxycarbonylpropyl, hydroxymethyl, hydroxyethyl, hydroxypropyl, hydroxybutyl, phenyl;
R⁹ and R¹⁰ are each independently selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, and C₆₋₁₂aryl; preferably R¹⁰ is hydrogen or C₁₋₆alkyl, and R⁹ is selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, C₆₋₁₂aryl; preferably R⁹ and R¹⁰ are each independently selected from the group consisting of H, C₁₋₆alkyl, C₁₋₄alkylaminoC₁₋₆alkyl, aminoC₁₋₆alkyl, cyclopropyl, cyclobutyl, cyclopentyl, cyclohexyl, C₁₋₄alkoxycarbonylC₁₋₆alkyl, hydroxyC₁₋₄alkyl, phenyl, naphthalenyl; preferably R¹⁰ is hydrogen or C₁₋₄alkyl, and R⁹ is selected from the group consisting of H, C₁₋₄alkyl, C₁₋₄alkylaminoC₁₋₄alkyl, aminoC₁₋₄alkyl, cyclopropyl, cyclobutyl, cyclopentyl, cyclohexyl, C₁₋₄alkoxycarbonylC₁₋₄alkyl, hydroxyC₁₋₄alkyl, phenyl; preferably R¹⁰ is hydrogen, and R⁹ is selected from the group consisting of H, methyl, ethyl, propyl, isopropyl, butyl, isobutyl, tert-butyl, C₁₋₄alkylaminoC₁₋₄alkyl, aminoC₁₋₄alkyl, cyclopropyl, cyclobutyl, cyclopentyl, cyclohexyl, C₁₋₄alkoxycarbonylC₁₋₄alkyl, hydroxymethyl, hydroxyethyl, hydroxypropyl, hydroxybutyl, phenyl; preferably R¹⁰ is hydrogen, and R⁹ is selected from the group consisting of H, methyl, ethyl, propyl, isopropyl, butyl, isobutyl, tert-butyl, C₁₋₄alkylaminomethyl, C₁₋₄alkylaminoethyl, C₁₋₄alkylaminopropyl, C₁₋₄alkylaminobutyl, aminomethyl, aminoethyl, aminopropyl, aminobutyl, cyclopropyl, cyclobutyl, cyclopentyl, cyclohexyl, C₁₋₄alkoxycarbonylmethyl, C₁₋₄alkoxycarbonylethyl, C₁₋₄alkoxycarbonylbutyl, C₁₋₄alkoxycarbonylpropyl, hydroxymethyl, hydroxyethyl, hydroxypropyl, hydroxybutyl, phenyl; preferably R¹⁰ is hydrogen, and R⁹ is selected from the group consisting of H, methyl, ethyl, propyl, isopropyl, butyl, isobutyl, tert-butyl, C₁₋₄alkylaminomethyl, C₁₋₄alkylaminoethyl, C₁₋₄alkylaminopropyl, C₁₋₄alkylaminobutyl, aminomethyl, aminoethyl, aminopropyl, aminobutyl, cyclopentyl, cyclohexyl, C₁₋₄alkoxycarbonylmethyl, C₁₋₄alkoxycarbonylethyl, C₁₋₄alkoxycarbonylbutyl, C₁₋₄alkoxycarbonylpropyl, hydroxymethyl, hydroxyethyl, hydroxypropyl, hydroxybutyl, phenyl;
R¹¹ and R¹² are each independently selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, and C₆₋₁₂aryl; preferably R¹³ is hydrogen or C₁₋₆alkyl, and R¹¹ is selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, C₆₋₁₂aryl; preferably R¹¹ and R¹² are each independently selected from the group consisting of H, C₁₋₆alkyl, C₁₋₄alkylaminoC₁₋₆alkyl, aminoC₁₋₆alkyl, cyclopropyl, cyclobutyl, cyclopentyl, cyclohexyl, C₁₋₄alkoxycarbonylC₁₋₆alkyl, hydroxyC₁₋₄alkyl, phenyl, naphthalenyl; preferably R¹² is hydrogen or C₁₋₄alkyl, and R¹¹ is selected from the group consisting of H, C₁₋₄alkyl, C₁₋₄alkylaminoC₁₋₄alkyl, aminoC₁₋₄alkyl, cyclopropyl, cyclobutyl, cyclopentyl, cyclohexyl, C₁₋₄alkoxycarbonylC₁₋₄alkyl, hydroxyC₁₋₄alkyl, phenyl; preferably R¹² is hydrogen, and R¹¹ is selected from the group consisting of H, methyl, ethyl, propyl, isopropyl, butyl, isobutyl, tert-butyl, C₁₋₄alkylaminoC₁₋₄alkyl, aminoC₁₋₄alkyl, cyclopropyl, cyclobutyl, cyclopentyl, cyclohexyl, C₁₋₄alkoxycarbonylC₁₋₄alkyl, hydroxymethyl, hydroxyethyl, hydroxypropyl, hydroxybutyl, phenyl; preferably R¹² is hydrogen, and R¹¹ is selected from the group consisting of H, methyl, ethyl, propyl, isopropyl, butyl, isobutyl, tert-butyl, C₁₋₄alkylaminomethyl, C₁₋₄alkylaminoethyl, C₁₋₄alkylaminopropyl, C₁₋₄alkylaminobutyl, aminomethyl, aminoethyl, aminopropyl, aminobutyl, cyclopropyl, cyclobutyl, cyclopentyl, cyclohexyl, C₁₋₄alkoxycarbonylmethyl, C₁₋₄alkoxycarbonylethyl, C₁₋₄alkoxycarbonylbutyl, C₁₋₄alkoxycarbonylpropyl, hydroxymethyl, hydroxyethyl, hydroxypropyl, hydroxybutyl, phenyl; preferably R¹² is hydrogen, and R¹¹ is selected from the group consisting of H, methyl, ethyl, propyl, isopropyl, butyl, isobutyl, tert-butyl, C₁₋₄alkylaminomethyl, C₁₋₄alkylaminoethyl, C₁₋₄alkylaminopropyl, C₁₋₄alkylaminobutyl, aminomethyl, aminoethyl, aminopropyl, aminobutyl, cyclopentyl, cyclohexyl, C₁₋₄alkoxycarbonylmethyl, C₁₋₄alkoxycarbonylethyl, C₁₋₄alkoxycarbonylbutyl, C₁₋₄alkoxycarbonylpropyl, hydroxymethyl, hydroxyethyl, hydroxypropyl, hydroxybutyl, phenyl;
R¹³ and R¹⁴ are each independently selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, and C₆₋₁₂aryl; preferably R¹⁴ is hydrogen or C₁₋₆alkyl, and R¹³ is selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, C₆₋₁₂aryl; preferably R¹³ and R¹⁴ are each independently selected from the group consisting of H, C₁₋₆alkyl, C₁₋₄alkylaminoC₁₋₆alkyl, aminoC₁₋₆alkyl, cyclopropyl, cyclobutyl, cyclopentyl, cyclohexyl, C₁₋₄alkoxycarbonylC₁₋₆alkyl, hydroxyC₁₋₄alkyl, phenyl, naphthalenyl; preferably R¹⁴ is hydrogen or C₁₋₄alkyl, and R¹³ is selected from the group consisting of H, C₁₋₄alkyl, C₁₋₄alkylaminoC₁₋₄alkyl, aminoC₁₋₄alkyl, cyclopropyl, cyclobutyl, cyclopentyl, cyclohexyl, C₁₋₄alkoxycarbonylC₁₋₄alkyl, hydroxyC₁₋₄alkyl, and phenyl; preferably R¹⁴ is hydrogen, and R¹³ is selected from the group consisting of H, methyl, ethyl, propyl, isopropyl, butyl, isobutyl, tert-butyl, C₁₋₄alkylaminoC₁₋₄alkyl, aminoC₁₋₄alkyl, cyclopropyl, cyclobutyl, cyclopentyl, cyclohexyl, C₁₋₄alkoxycarbonylC₁₋₄alkyl, hydroxymethyl, hydroxyethyl, hydroxypropyl, hydroxybutyl, phenyl; preferably R¹⁴ is hydrogen, and R¹³ is selected from the group consisting of H, methyl, ethyl, propyl, isopropyl, butyl, isobutyl, tert-butyl, C₁₋₄alkylaminomethyl, C₁₋₄alkylaminoethyl, C₁₋₄alkylaminopropyl, C₁₋₄alkylaminobutyl, aminomethyl, aminoethyl, aminopropyl, aminobutyl, cyclopropyl, cyclobutyl, cyclopentyl, cyclohexyl, C₁₋₄alkoxycarbonylmethyl, C₁₋₄alkoxycarbonylethyl, C₁₋₄alkoxycarbonylbutyl, C₁₋₄alkoxycarbonylpropyl, hydroxymethyl, hydroxyethyl, hydroxypropyl, hydroxybutyl, phenyl; preferably R¹⁴ is hydrogen, and R¹³ is selected from the group consisting of H, methyl, ethyl, propyl, isopropyl, butyl, isobutyl, tert-butyl, C₁₋₄alkylaminomethyl, C₁₋₄alkylaminoethyl, C₁₋₄alkylaminopropyl, C₁₋₄alkylaminobutyl, aminomethyl, aminoethyl, aminopropyl, aminobutyl, cyclopentyl, cyclohexyl, C₁₋₄alkoxycarbonylmethyl, C₁₋₄alkoxycarbonylethyl, C₁₋₄alkoxycarbonylbutyl, C₁₋₄alkoxycarbonylpropyl, hydroxymethyl, hydroxyethyl, hydroxypropyl, hydroxybutyl, phenyl;
R¹⁵ and R⁸ are each independently selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, and C₆₋₁₂aryl; preferably R¹⁶ is hydrogen or C₁₋₆alkyl, and R¹⁵ is selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, C₆₋₁₂aryl; preferably R¹⁵ and R¹⁶ are each independently selected from the group consisting of H, C₁₋₆alkyl, C₁₋₄alkylaminoC₁₋₆alkyl, aminoC₁₋₆alkyl, cyclopropyl, cyclobutyl, cyclopentyl, cyclohexyl, C₁₋₄alkoxycarbonylC₁₋₆alkyl, hydroxyC₁₋₄alkyl, phenyl, naphthalenyl; preferably R¹⁶ is hydrogen or C₁₋₄alkyl, and R¹⁵ is selected from the group consisting of H, C₁₋₄alkyl, C₁₋₄alkylaminoC₁₋₄alkyl, aminoC₁₋₄alkyl, cyclopropyl, cyclobutyl, cyclopentyl, cyclohexyl, C₁₋₄alkoxycarbonylC₁₋₄alkyl, hydroxyC₁₋₄alkyl, phenyl; preferably R¹⁶ is hydrogen, and R¹⁵ is selected from the group consisting of H, methyl, ethyl, propyl, isopropyl, butyl, isobutyl, tert-butyl, C₁₋₄alkylaminoC₁₋₄alkyl, aminoC₁₋₄alkyl, cyclopropyl, cyclobutyl, cyclopentyl, cyclohexyl, C₁₋₄alkoxycarbonylC₁₋₄alkyl, hydroxymethyl, hydroxyethyl, hydroxypropyl, hydroxybutyl, phenyl; preferably R¹⁶ is hydrogen, and R¹⁵ is selected from the group consisting of H, methyl, ethyl, propyl, isopropyl, butyl, isobutyl, tert-butyl, C₁₋₄alkylaminomethyl, C₁₋₄alkylaminoethyl, C₁₋₄alkylaminopropyl, C₁₋₄alkylaminobutyl, aminomethyl, aminoethyl, aminopropyl, aminobutyl, cyclopropyl, cyclobutyl, cyclopentyl, cyclohexyl, C₁₋₄alkoxycarbonylmethyl, C₁₋₄alkoxycarbonylethyl, C₁₋₄alkoxycarbonylbutyl, C₁₋₄alkoxycarbonylpropyl, hydroxymethyl, hydroxyethyl, hydroxypropyl, hydroxybutyl, phenyl; preferably R¹⁶ is hydrogen, and R¹⁵ is selected from the group consisting of H, methyl, ethyl, propyl, isopropyl, butyl, isobutyl, tert-butyl, C₁₋₄alkylaminomethyl, C₁₋₄alkylaminoethyl, C₁₋₄alkylaminopropyl, C₁₋₄alkylaminobutyl, aminomethyl, aminoethyl, aminopropyl, aminobutyl, cyclopentyl, cyclohexyl, C₁₋₄alkoxycarbonylmethyl, C₁₋₄alkoxycarbonylethyl, C₁₋₄alkoxycarbonylbutyl, C₁₋₄alkoxycarbonylpropyl, hydroxymethyl, hydroxyethyl, hydroxypropyl, hydroxybutyl, phenyl;
R¹⁷ and R¹⁸ are each independently selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, and C₆₋₁₂aryl; preferably R¹³ is hydrogen or C₁₋₆alkyl, and R¹⁷ is selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, C₆₋₁₂aryl; preferably R¹⁷ and R¹⁸ are each independently selected from the group consisting of H, C₁₋₆alkyl, C₁₋₄alkylaminoC₁₋₆alkyl, aminoC₁₋₆alkyl, cyclopropyl, cyclobutyl, cyclopentyl, cyclohexyl, C₁₋₄alkoxycarbonylC₁₋₆alkyl, hydroxyC₁₋₄alkyl, phenyl, naphthalenyl; preferably R¹⁸ is hydrogen or C₁₋₄alkyl, and R¹⁷ is selected from the group consisting of H, C₁₋₄alkyl, C₁₋₄alkylaminoC₁₋₄alkyl, aminoC₁₋₄alkyl, cyclopropyl, cyclobutyl, cyclopentyl, cyclohexyl, C₁₋₄alkoxycarbonylC₁₋₄alkyl, hydroxyC₁₋₄alkyl, phenyl; preferably R¹⁸ is hydrogen, and R¹⁷ is selected from the group consisting of H, methyl, ethyl, propyl, isopropyl, butyl, isobutyl, tert-butyl, C₁₋₄alkylaminoC₁₋₄alkyl, aminoC₁₋₄alkyl, cyclopropyl, cyclobutyl, cyclopentyl, cyclohexyl, C₁₋₄alkoxycarbonylC₁₋₄alkyl, hydroxymethyl, hydroxyethyl, hydroxypropyl, hydroxybutyl, phenyl; preferably R¹⁸ is hydrogen, and R¹⁷ is selected from the group consisting of H, methyl, ethyl, propyl, isopropyl, butyl, isobutyl, tert-butyl, C₁₋₄alkylaminomethyl, C₁₋₄alkylaminoethyl, C₁₋₄alkylaminopropyl, C₁₋₄alkylaminobutyl, aminomethyl, aminoethyl, aminopropyl, aminobutyl, cyclopropyl, cyclobutyl, cyclopentyl, cyclohexyl, C₁₋₄alkoxycarbonylmethyl, C₁₋₄alkoxycarbonylethyl, C₁₋₄alkoxycarbonylbutyl, C₁₋₄alkoxycarbonylpropyl, hydroxymethyl, hydroxyethyl, hydroxypropyl, hydroxybutyl, phenyl; preferably R¹⁸ is hydrogen, and R¹⁷ is selected from the group consisting of H, methyl, ethyl, propyl, isopropyl, butyl, isobutyl, tert-butyl, C₁₋₄alkylaminomethyl, C₁₋₄alkylaminoethyl, C₁₋₄alkylaminopropyl, C₁₋₄alkylaminobutyl, aminomethyl, aminoethyl, aminopropyl, aminobutyl, cyclopentyl, cyclohexyl, C₁₋₄alkoxycarbonylmethyl, C₁₋₄alkoxycarbonylethyl, C₁₋₄alkoxycarbonylbutyl, C₁₋₄alkoxycarbonylpropyl, hydroxymethyl, hydroxyethyl, hydroxypropyl, hydroxybutyl, phenyl;
M is selected from the group consisting of Li, Na, K, Cs, Rb; preferably M is selected from Li, Na or K, preferably M is selected from Na or K, preferably M is Na.

One embodiment of the invention is a compound of formula (I), wherein R² is hydrogen, and R¹, R³, R⁴, R⁵, R⁶ have the meaning as defined above.

One embodiment of the invention is a compound of formula (I), wherein R³ is hydrogen and R¹, R², R⁵ and R⁶ have the meaning as defined above.

One embodiment of the invention is a compound of formula (I), wherein R² and R³ are each independently hydrogen and R¹, R⁴, R⁵ and R⁶ have the meaning as defined above.

One embodiment of the invention is a compound of formula (I), wherein R², R³ and R⁴ are each independently hydrogen and R¹, R⁵ and R⁶ have the meaning as defined above.

One embodiment of the invention is a compound of formula (I), wherein R¹ and R⁶ are the same.

One embodiment of the invention is a compound of formula (I), wherein R² and R³ are the same.

Another embodiment of the invention is a compound having the structural formula (II)

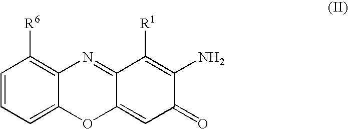

wherein R¹ and R⁶ have the meaning defined above.

According to one aspect of the invention is the compound having the structural formula (II), wherein

R¹ is selected from a group consisting of —SO₃H, —SO₃M, —SO₂NR⁷R⁸, —SO₂—C₁₋₆alkyl, —SO₂—C₆₋₁₂aryl, —SO₂O—C₁₋₆alkyl, and —SO₂O—C₆₋₁₂aryl;
preferably R¹ is selected from —SO₂NR⁷R⁸, —SO₂-phenyl, or —SO₂O-phenyl;
preferably R¹ is selected from —SO₂H, —SO₂—C₁₋₆alkyl, or —SO₂O—C₁₋₄alkyl;
preferably R¹ is —SO₂NR⁷R⁸;
preferably R¹ is selected from —SO₃H, —SO₂NHphenyl, —SO₂NH₂, —SO₂NHC₄₋₆cycloalkyl, —SO₂NHC₁₋₆alkyleneN(C₁₋₆alkyl)₂, —SO₂NHC₁₋₆alkyleneNH₂, —SO₂NHC₁₋₆alkylene-C(═O)O—C₁₋₆alkyl, or —SO₂NHC₁₋₆alkylene-OH;
preferably R¹ is selected from —SO₂NHphenyl, —SO₂NH₂, —SO₂NHcyclohexyl, —SO₂NHC₃alkyleneN(methyl)₂, —SO₂NHethyleneNH₂, —SO₂NHmethylene-C(═O)O—C₁₋₆alkyl, or —SO₂NHmethylene-OH;
preferably R¹ is —SO₃H;
R⁶ is selected from the group consisting of —SO₃H, —SO₃M, —SO₂NR¹⁷R¹⁸, —SO₂—C₁₋₆alkyl, —SO₂—C₆₋₁₂aryl, —SO₂O—C₁₋₆alkyl, and —SO₂O—C₆₋₁₂aryl, R², R³, R⁴ and R⁵ are hydrogen atoms,
preferably R⁶ is selected from the group consisting of —SO₃H, —SO₃M, —SO₂NR¹⁷R¹⁸, —SO₂H, —SO₂—C₁₋₆alkyl, —SO₂-phenyl, —SO₂O—C₁₋₄alkyl, —SO₂O-phenyl, C₁₋₆alkyl, C₆₋₁₂aryl, SH, OH, —NH₂, halogen, carboxyl, aminocarbonyl, C₁₋₆alkylaminocarbonyl, and C₆₋₁₂arylaminocarbonyl;
preferably R⁶ is selected from —SO₂NR¹⁷R¹⁸, —SO₂-phenyl, or —SO₂O-phenyl;
preferably R⁶ is selected from —SO₂H, —SO₂—C₁₋₆alkyl, or —SO₂O—C₁₋₄alkyl;
preferably R⁶ is —SO₂NR¹⁷R¹⁸;
preferably R⁶ is selected from —SO₃H, —SO₂NHphenyl, —SO₂NH₂, —SO₂NHC₄₋₆cycloalkyl, —SO₂NHC₁₋₆alkyleneN(C₁₋₆alkyl)₂, —SO₂NHC₁₋₆alkyleneNH₂, —SO₂NHC₁₋₆alkylene-C(═O)O—C₁₋₆alkyl, or —SO₂NHC₁₋₆alkylene-OH; preferably R⁶ is selected from —SO₂NHphenyl, —SO₂NH₂, —SO₂NHcyclohexyl, —SO₂NHC₃alkyleneN(methyl)₂, —SO₂NHethyleneNH₂, —SO₂NHmethylene-C(═O)O—C₁₋₆alkyl, or —SO₂NHmethylene-OH; preferably R³ is —SO₃H; and
R¹⁷ and R¹⁸ have the meaning as defined above.

One embodiment of the invention is a compound of formula (II), wherein R¹ and R⁶ are the same.

In a preferred embodiment, the present invention encompasses compounds of formula (I) wherein R¹ is —SO₂NR⁷R⁸; R⁶ is —SO₂NR¹⁷R¹⁸,

R⁷ and R⁸ are each independently selected from the group consisting of hydrogen, C₁₋₆alkyl, C₁₋₆alkylaminoC₁₋₆alkyl, aminoC₁₋₆alkyl, C₃₋₆cycloalkyl, C₁₋₆alkoxycarbonylC₁₋₆alkyl, hydroxyC₁₋₆alkyl, and C₆₋₁₀aryl;
R¹⁷ and R¹⁸ are each independently selected from the group consisting of hydrogen, C₁₋₆alkyl, C₁₋₆alkylaminoC₁₋₆alkyl, aminoC₁₋₆alkyl, C₃₋₆cycloalkyl, C₁₋₆alkoxycarbonylC₁₋₆alkyl, hydroxyC₁₋₆alkyl, and C₆₋₁₀aryl; and
R², R³, R⁴ and R⁵ are each independently hydrogen.

In a preferred embodiment, the present invention encompasses compounds of formula (I) wherein R¹ is —SO₂NR⁷R⁸; R⁶ is —SO₂NR¹⁷R¹⁸,

R⁷ is selected from the group consisting of hydrogen, C₁₋₆alkyl, C₁₋₆alkylaminoC₁₋₆alkyl, aminoC₁₋₆alkyl, C₃₋₆cycloalkyl, C₁₋₆alkoxycarbonylC₁₋₆alkyl, hydroxyC₁₋₆alkyl, and C₆₋₁₀aryl;
R⁸ is hydrogen,
R¹⁷ is selected from the group consisting of hydrogen, C₁₋₆alkyl, C₁₋₆alkylaminoC₁₋₆alkyl, aminoC₁₋₆alkyl, C₃₋₆cycloalkyl, C₁₋₆alkoxycarbonylC₁₋₆alkyl, hydroxyC₁₋₆alkyl, and C₆₋₁₀aryl;
R¹⁸ is hydrogen, and R², R³, R⁴ and R⁵ are each independently hydrogen.

In a preferred embodiment, the present invention encompasses compounds of formula (I) wherein R¹ is —SO₂NR⁷R⁸; R⁶ is —SO₂NR¹⁷R¹⁸,

R⁷ is selected from the group consisting of hydrogen, C₁₋₅alkyl, C₁₋₅alkylaminoC₁₋₅alkyl, aminoC₁₋₅alkyl, C₃₋₆cycloalkyl, C₁₋₄alkoxycarbonylC₁₋₄alkyl, hydroxyC₁₋₄alkyl, and phenyl;
R⁸ is hydrogen,
R¹⁷ is selected from the group consisting of hydrogen, C₁₋₅alkyl, C₁₋₅alkylaminoC₁₋₅alkyl, aminoC₁₋₅alkyl, C₃₋₆cycloalkyl, C₁₋₄alkoxycarbonylC₁₋₄alkyl, hydroxyC₁₋₄alkyl, and phenyl;
R¹⁸ is hydrogen, and R², R³, R⁴ and R⁵ are each independently hydrogen.

Another embodiment of the invention is a compound having the structural formula (III)

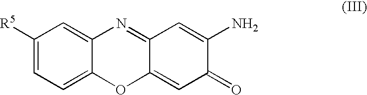

wherein R⁵ has the same meaning as defined above.

One aspect of the invention is the compound having the structural formula (III), wherein

R⁵ is selected from a group consisting of —SO₃H, —SO₃M, —SO₂NR¹⁵R¹⁶, —SO₂—C₁₋₆alkyl, —SO₂—C₆₋₁₂aryl, —SO₂O—C₁₋₆alkyl, and —SO₂O—C₆₋₁₂aryl, preferably —SO₃H, —SO₃M, —SO₂NR¹⁵R¹⁶, —SO₂H, —SO₂—C₁₋₆alkyl, —SO₂-phenyl, —SO₂O—C₁₋₄alkyl, —SO₂O-phenyl, C₁₋₆alkyl, C₆₋₁₂aryl, SH, OH, —NH₂, halogen, carboxyl, aminocarbonyl, C₁₋₆alkylaminocarbonyl, C₆₋₁₂arylaminocarbonyl; preferably R⁵ is selected from —SO₂NR¹⁵R¹⁶, —SO₂-phenyl, —SO₂O-phenyl; preferably R⁵ is selected from —SO₂H, —SO₂—C₁₋₆alkyl, —SO₂O—C₁₋₄alkyl; preferably R⁵ is selected from —SO₂NR¹⁵R¹⁶; preferably R⁵ is selected from —SO₃H, —SO₂NHphenyl, —SO₂NH₂, —SO₂NHC₄₋₆cycloalkyl, —SO₂NHC₁₋₆alkyleneN(C₁₋₆alkyl)₂, —SO₂NHC₁₋₆alkyleneNH₂, —SO₂NHC₁₋₆alkylene-C(═O)O—C₁₋₆alkyl, —SO₂NHC₁₋₆alkylene-OH;
preferably R⁵ is selected from —SO₂NHphenyl, —SO₂NH₂, —SO₂NHcyclohexyl, —SO₂NHC₃alkyleneN(methyl)₂, —SO₂NHethyleneNH₂, —SO₂NHmethylene-C(═O)O—C₁₋₆alkyl, or —SO₂NHmethylene-OH; preferably R⁵ is —SO₃H, and
R¹⁵ and R¹⁶ have the meaning as defined above.

The compounds of formula (I), (II) and (III) may be prepared according to a method of the invention using the enzyme laccase as an oxidant catalyst for the regioselective dimerization of an aromatic precursor. Preferably, the aromatic precursor is a substituted 3-Hydroxy-2-amino-benzene or a substituted 4-Hydroxy-3-amino-benzene compound. Most preferably, the aromatic precursor has the formula (IV):

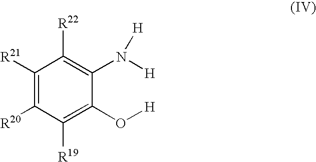

where R¹⁹ is the same as R² or R³ as defined above, R²⁰ is the same as R⁴ as defined above, R²¹ is the same as R⁵ as defined above, and R²² is the same as R¹ or R⁶ as defined above.

In an embodiment, the catalytic reaction oxidatively dimerises the aromatic precursor (IV) to form compounds of the invention having formula (I), as shown in Scheme 1.

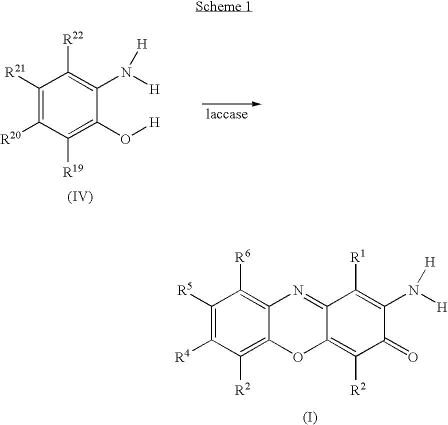

The product may be readily purified from the reaction using the methods described herein.

One aspect of the invention is a method described herein, wherein R²² is the same as R¹ or R⁶ as defined above, and R¹⁹, R²⁰, and R²¹ are hydrogen atoms. Another aspect of the invention is a method described herein, wherein R²² is —SO₃H, and R¹⁹, R²⁰, and R²¹ are hydrogen atoms. Another aspect of the invention is a method described herein, wherein the compound of formula (IV) is any of compounds 3 to 7 listed in Table 1. Another aspect of the invention is a method described herein, wherein R²¹ is the same as R⁵ as above, and R¹⁹, R²⁰, and R²² are hydrogen atoms. One aspect of the invention is a method described herein, wherein R²¹ is —SO₃H, and R¹⁹, R²⁰, and R²² are hydrogen atoms.

A laccase enzyme as used by the present invention refers to an enzyme belonging to the lacasse class (EC 1.10.3.2). There are benzenediol:oxygen oxidoreductases that oxidise a wide variety of organic compounds, causing O- and N-demethylation reactions, carbon-carbon bond cleavage or polymerizations (Giurg, et al. 2007. Synthetic Communications 37:1779-1789). These biocatalysts require molecular oxygen as the electron acceptor therefore producing water (Shaw, S. D., and H. S. Freeman. 2004. Textile Research Journal 74:215-222). This co-substrate is easily renewed in the reaction medium by running the experiment in aerated and agitated vessels. This represents an advantage for industrial applications. Moreover, laccases can be cheaply available as they may be secreted by some fungi in high level upon induction. According to one aspect of the invention, the laccase enzyme is that produced by a white rot fungus.

One embodiment of the present invention is a method for the preparation of a compound of formula (I) comprising the step of incubating a laccase enzyme with an aromatic precursor having the formula (IV), which enzyme catalyses a regioselective dimerization of the aromatic precursor, thereby producing a compound of formula (I).

In a preferred embodiment of the invention, formation of the compounds of formula (I) by biocatalysis proceeds in an aqueous medium comprising water (e.g. ultrapure, deionised water) and an aqueous buffer such as phosphate buffer or ammonium buffer. The medium may alternatively comprise water, being adjusted to a suitable pH by the addition of strong acid and base such as hydrochloric acid and sodium hydroxide. The reaction medium may further comprise an alcohol (e.g. C₁₋₄ alcohol, such as ethanol, methanol, propanol, butanol) at a concentration (v/v) of 0.25%, 0.5%, 1%, 2%, 3%, 4%, 5%, 6%, 7%, 8%, 9%, 10%, or a value in the range between any two of the aforementioned values, preferably between 1% and 10%. The medium contains the appropriate substrate (e.g. compound IV), at a concentration equal to or less than 1 g·L⁻¹, 2 g·L⁻¹, 3 g·L⁻¹, 4 g·L⁻¹, 5 g·L⁻¹, 6 g·L⁻¹, 7 g·L⁻¹, 8 g·L⁻¹, 9 g·L⁻¹, 10 g·L⁻¹, 10 g·L⁻¹, 11 g·L⁻¹, 12 g·L⁻¹, 13 g·L⁻¹, 14 g·L⁻¹, 15 g·L⁻¹, 16 g·L⁻¹, 17 g·L⁻¹, 18 g·L⁻¹, 19 g·L⁻¹, or 20 g·L⁻¹, or a value in the range between any two of the aforementioned values, preferably between 1 and 10 g·L⁻¹.

The medium also comprises a quantity of laccase either in solution or immobilized on a suitable support such as perlite, glass beads, so that the resulting activity of the enzyme in the solution is equal to or greater than 1, 10, 50, 100, 150, 200, 250, 300, 400, 500, 600, 700, 800, 900, 1000, 1100, 1150, 1200, 1250, 1300, 1400, 1500, 1600, 1700, 1800, 1900, or 2000 U·L⁻¹ or a value in the range between any two of the aforementioned values, preferably between 1 and 1000 U·L⁻¹.

The reaction is preferably performed aerobically using common glassware (e.g. beaker or flask) agitated by magnetic stirrer, or by agitation in shaking flasks or using a fermentor, if appropriate with the introduction of air or oxygen.

The enzymatic reaction may be carried at a temperature ranging from about 18° to 35° C., preferably at about 25° to 30° C. The reaction is carried out in a pH range from 2 to 8, preferably between 5 and 6. Under these conditions, the reaction medium in general shows a noticeable to complete accumulation of the desired compound after 5 to 24 hours.

Another embodiment of the present invention is a method for the preparation of a compound of formula (I) comprising the step of incubating a microorganism containing a gene for laccase enzyme expression with an aromatic precursor having the formula (IV), which enzyme catalyses a regioselective dimerization of the aromatic precursor, thereby producing a compound of formula (I).

Thus, instead of employing a purified laccase enzyme, cells which express and optionally secrete laccase may be utilised in fermentation conditions to convert the substrate. Suitable cells include bacterial cells transformed with a plasmid construct encoding the laccase gene provided with a suitable promotor, which construct is obtained using methods well known in the art, for instance, as described in “Molecular Cloning—A Laboratory Manual” (Maniatis, Sambrook, Fritsch, Cold Spring Harbor Laboratory Press; 2nd edition). Suitable cells that natively express laccase include fungal (mycelium) cells, such as those from the dry rot fungus, particularly from Cerrena unicolor. The skilled person would be able to utilise practices well known in the art to prepare and purify products of the invention from the fermentation medium. As an example, Cerrena unicolor may be used to inoculate a medium containing between 20 and 30 g·L⁻¹ potato dextrose broth. The medium is grown at a temperature between 20 and 30 deg C., which medium contains the substrate. The product—a compound of the invention—is continually formed in the medium, and can be extracted from the regions surrounding the mycelium. By continually replenishing the medium and substrate, and continually extracting product-containing medium, a continuous flow synthesis is achieved.

The course of the reaction can be monitored with the aid of the pH measurement of the medium, the aid of HPLC method or by thin layer chromatography. The compounds of interest which can be prepared by biotransformation are isolated from the medium of reaction by known methods, taking into account the chemical, physical and biological properties of the products.

The skilled person will appreciate that reaction products and unreacted reagents may be detected by thin layer chromatography on silica gel with polar solvent mixture, for example n-butanol/acetic acid/water as the mobile phase. Alternatively the detection can be carried out by known liquid chromatographic methods (e.g. HPLC).

The compound can be further purified by appropriate filtration through a plug of reversed phase silica gel with water as the mobile phase up to a purity of about 90%. Further purification can be accomplished by known methods, for example, using semi-preparative HPLC.

The present invention also concerns a use of a compound of the invention as a dye. The dye is convenient to dye textile, leather wherein said material is a fabric, yarn, fiber, garment or film made of a material selected from the group consisting of fur, hide, leather, silk, wool, cationic polysaccharide, synthetic polyamide. The dye is also useful for dying hair.

The dye according to the invention particularly provides an even colouration on acetate, cotton, nylon, PES, acrylic and wool keratin, fibres with favorable dyeing properties such as good fastness to light, washing, rubbing and perspiration.

The dyes of the formula (I), (II) or (III) according to the invention are preferably present in the colourants in a total amount of from 0.01 to 10 percent by weight, in particular 0.1 to 8 percent by weight.

To produce special colour shades, besides the dyes of the formula (I), (II) or (III) according to the invention, it is possible to add to the agents according to the invention one or more additional customary direct dye from the group consisting of acidic dyes, basic dyes, nitro dyes, azo dyes, anthraquinone dyes and triphenylmethane dyes. In addition, the colourants according to the invention can also comprise naturally occurring dyes, such as, for example, henna red, henna neutral, henna black, camomile, sandalwood, black tea, buckthorn bark, sage, logwood, madder root, catechu, sedre and alkanna root, particularly useful for the formulation of hair dyes.

The abovementioned additional direct dyes and naturally occurring dyes may be present in a total amount of from about 0.01 to 5 percent by weight, the total content of dyes in the colourant according to the invention being preferably from about 0.01 to 10 percent by weight, in particular 0.1 to 5 percent by weight.

### EXAMPLES

The invention is illustrated by way of the following non-limiting experimental examples.

### A) Preparation of Aromatic Precursors Having Formula (IV)

3-Hydroxy-orthanilic acid (3-HOA) was prepared according to Bruyneel et al. (Bruyneel et al., Eur. J. Org. Chem. 2008, 72-79). Compounds of formula (IV) can be prepared as illustrated in scheme 2. The derived sulfonamides could be obtained from 2-t-butylbenzoxazole-4-sulfonic acid (1), an intermediate compound of the 3-HOA synthesis. This compound was transformed into the corresponding sulfonyl chloride (2) (PCl₅—OPCl₃, CH₂Cl₂, 40° C., 2 h) which reacted with ammonia and R-substituted amines to furnish sulfonamides (formula V), unsubstituted and N-substituted respectively (NH₄OH excess or RNH₂ 10% water, 20° C., 17 h). The so-obtained 2-t-butylbenzoxazole-4-sulfonamides (formula V) were hydrolyzed (HCl 6N, 90° C., 17 h) into 3-hydroxy-2-amino-benzene sulfonamides (formula VI).

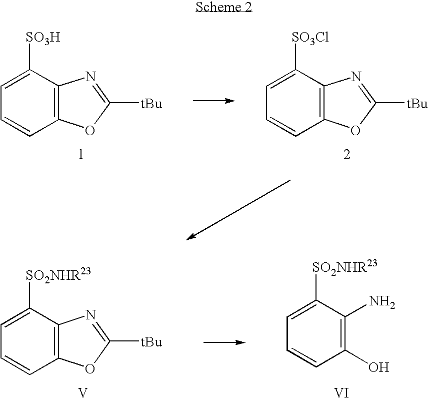

The structural formulas of the precursor of formula VI prepared are listed in Table 1.

TABLE 1
Structural formulas of compounds 3 to 9 synthesised according
to a method of example A.

| | |
|---|---|
| | (VI) |
| 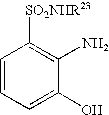 | |

| R²³ | Precursors and corresponding compound number |
|---|---|
| H | 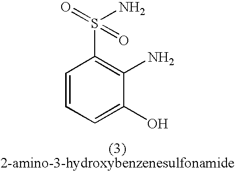 |
| Phenyl | 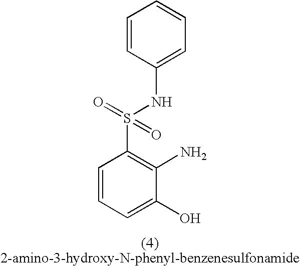 |
| C₆H₁₁ | 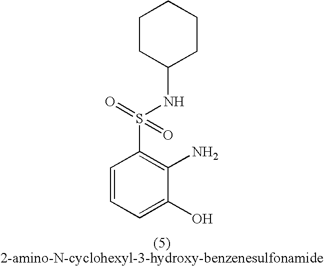 |
| (CH₂)₃—N(CH₃)₂ | 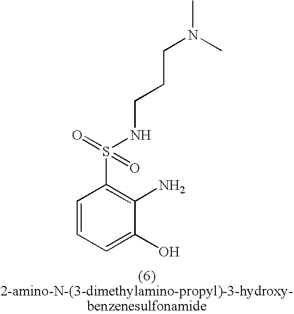 |
| CH₂—CH₂—NH₂ | 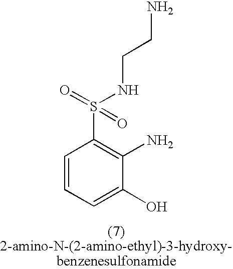 |
| CH₂—CO₂CH₃ | 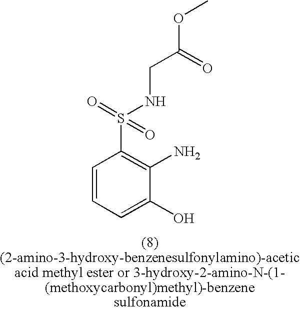 |
| (CH₂)₃—OH | 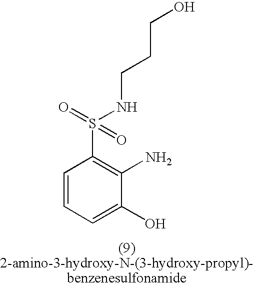 |

Each precursor (compound 3 to 9) was characterised by standard spectroscopic techniques including IR, ¹H NMR, ¹³C NMR, Mass, UV.

### B) Catalysed Oxidative Coupling

3-Hydroxy-2-amino-benzene sulfonamide compounds 3 to 9 as indicated in Table 1 were submitted to the laccase processing under reaction conditions as follows. A solution of laccase (200 UL⁻¹) and at least one precursor (5.29 mmol) in a final volume of 100 mL of water adjusted at pH 6 with HCl and NaOH, was prepared. The stirred mixture was kept 24 h at 25° C. The reaction was stopped by decreasing the pH with MSA (pH˜2). The crude mixture was freeze dried to give a powder. Part of this powder was purified by chromatography on reversed phase (water as eluant) to yield an almost pure fraction of compound. Part of this fraction was submitted to preparative HPLC to yield pure title compound. Yield of about 70% was obtained after freeze drying, The yield determined by HPLC. The products obtained (compounds 10 to 16 as indicated in Table 2 below) were isolated by HPLC and characterized by the usual spectroscopic techniques (IR, ¹H NMR, ¹³C NMR, Mass, UV).

TABLE 2
List of precursors (3 to 9) and corresponding products (10 to 16)
prepared according to methods according to embodiments of the
present invention.

| Precursor | Product structure and compound number |
|---|---|
| 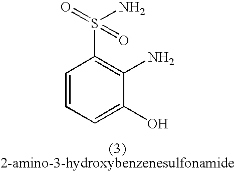 | 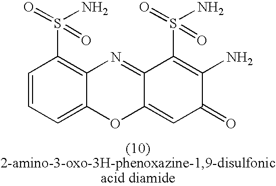 |
| 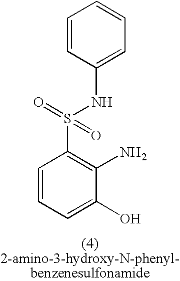 | 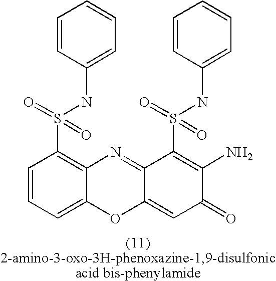 |
| 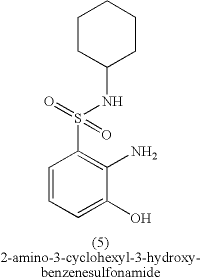 | 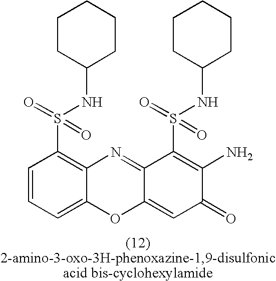 |
| 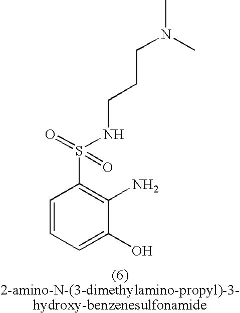 | 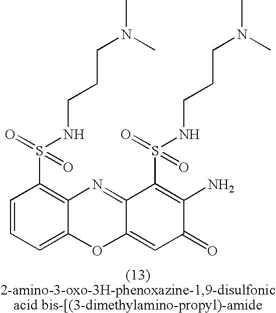 |
| 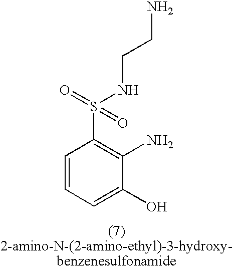 | 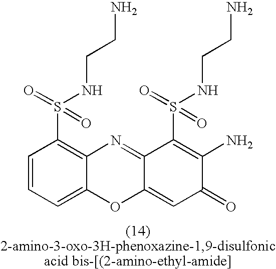 |
| 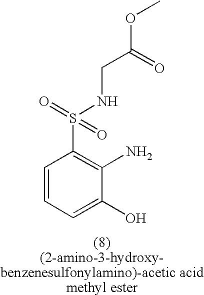 | 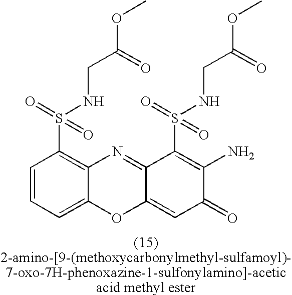 |
| 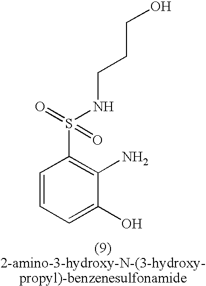 | 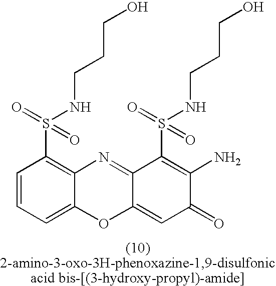 |

### C) Preparation of compound 17, 2-Amino-3oxo-3H-phenoxazine-1,9-disulfonic acid

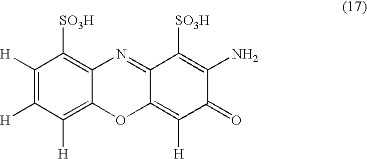

A particular compound of the invention, compound 17, an instance of a compound of structural formula (II), was prepared by dimerisation of the aromatic precursor that is 2-amino-3-hydroxybenzenesulfonic acid as follows. A solution of laccase (200 U L⁻¹) and 2-amino-3-hydroxybenzenesulfonic acid (5.29 mmol; 1.02 g) in a final volume of 100 mL of water adjusted at pH 6 with HCl and NaOH was prepared. The stirred mixture was kept 24 h at 25° C. The reaction was stopped by decreasing the pH with MSA (pH˜2). The crude mixture was freeze dried to give a red powder (1 g). Part of this powder (400 mg) was purified by chromatography on reversed phase (water as eluant) to yield an almost pure fraction of compound 17 (270 mg).

Part of this fraction (70 mg) was submitted to preparative HPLC to yield pure title compound as a deep red solid (30 mg). Yield of about 70% after freeze drying as determined by HPLC. The product was characterised as follows:

¹H NMR 500 MHz (DMSO) δ=9.81 (bs, 1H, NH), 9.45 (bs, 1H, NH), 7.74 (d, J_{H,H}=7.5 Hz, 1H, Ar—H), 7.58 (d, J_{H,H}=6.9 Hz, 1H, Ar—H), 7.5 (dd, J_{H,H}=7.5 Hz & 6.9 Hz, 1H, Ar—H) 6.65 (s, 1H) ppm.

¹³C NMR 75 MHz (DMSO) δ=176.3, 153.7, 150.1, 141.6, 139, 136.8, 127, 123.8, 123.2, 118.3, 117, 105 ppm.

MW: C₁₂H₈N₂O₈S₂, 372, 3305 g/mol. HRMS-ESI negative mode: m/z calcld 370.9644 for C₁₂H₇N₂O₈S₂. found: 370.9641. ESI-MS: (m/z) (%)=371.16 [M-H) (100); 393.1 [M-H+Na] (40); 291.2 (20).

M.p.>300° C.

IR (KBr): υ=3433, 2364, 1635, 1593, 1473, 1415, 1218, 1053.

UV: λₘₐₓ at 435 and 339 nm (0.1 M phosphate buffer, pH 7). 435 nm (ε=13981 L mol⁻¹ cm⁻¹).

UV: λₘₐₓ at 460 and 330 nm (0.1 M phosphate buffer, pH 2). 460 nm (ε=5426 L mol⁻¹ cm⁻¹).

Compound 17 has an orange-red colour. It has favourable staining properties, especially colour fastness, making it highly suitable for colouring textiles, hair and other articles.

### D) Preparation of the compound 18, 2-Amino-3oxo-3H-phenoxazine-8-sulfonic acid using whole cell synthesis

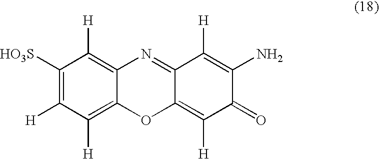

A particular compound of the invention, compound 18, an instance of a compound of structural formula (III), was prepared by dimerisation of the aromatic precursor 3-amino-4-hydroxybenzenesulfonic acid as follows.

### Inoculum Preparation and Immobilization of Mycelium:

Cerrena unicolor strain was precultured on Petri dish containing 2% (w/v) malt agar, incubated for 7 days at 24° C.+/−2° C. Four fragments of the mycelia were sampled at the margin of the colonies with a hollow-punch of 5 mm diameter and used to inoculate 100 ml of potato dextrose broth medium (2.4 g) in 250-ml Erlenmeyer flask. Cultures were incubated at a temperature of 24° C.+/−2° C. for 7 days, in stationary conditions. After this time, the whole content of the flasks was homogenized and the mycelium was used for the inoculation (10% v/v) of 500 ml water containing 24 g of potato dextrose broth in 1000-ml Erlenmeyer flask containing 2-3 rings (9 cm diameter and 3.5 cm high) of plastic mesh scourer. The submerged culture was incubated at 24° C.+/−2° C. during the next 5-7 days, in shaking conditions (140 rpm+/−20 rpm).

### The Substrate Transformation:

After overgrowing of carrier rings by mycelium (5-7 days), whole growing medium was repeated by addition of 500 ml of water (adjusted at pH 5 from strong NaOH) containing 2 g of 3-amino-4-hydroxybenzenesulfonic acid as the substrate for producing compound 18. Submerged culture was incubated for 48 hours at 24° C.+/−2° C., in shaking conditions (140 rpm+/−20 rpm). After 48 hours, coloured solution containing compound 18 was taken off from above immobilized mycelium, filtered off and, after freeze-drying, brown powder comprising compound 18 was obtained. After taking off the coloured solution from above the immobilized mycelium in the 1000-ml flask, a subsequent amount of water (500 ml, adjusted at pH 5 from strong NaOH) containing 2 g of 3-amino-4-hydroxybenzenesulfonic acid as the substrate for compound 18 was added. After a further 48 hours, coloured solution containing compound 18 was taken off from above the immobilized mycelium, filtered off, and after freeze drying, a brown powder of the compound 18 was obtained. In these sterile conditions it was possible to transform from 4 to 6 subsequent batches of 3-amino-4-hydroxybenzenesulfonic acid as the substrate for producing compound 18. After the 6^{th} batch, 500 ml of water containing 2.4 g of potato dextrose broth (refreshing medium) was added to immobilize the mycelium on the plastic mesh scourer rings for their refreshing and reinforcing. The culture was incubated for a further 48 hours at 24° C.+/−2° C., in shaking conditions (140 rpm+/−20 rpm). After a further 48 hours, whole refreshing of the medium was repeated by addition of 500 ml of water (adjusted at pH 5 from strong NaOH) containing 2 g of 3-amino-4-hydroxybenzenesulfonic acid as the substrate for producing compound 18.

### E) Preparation of the compound 18, 2-amino-3-oxo-3H-phenoxazine-8-sulfonic acid using isolated laccase

### Determination of Laccase Activity:

Standard laccase activity was determined by oxidation of 2.5 mM ABTS [2′,2-azino-bis-(3-ethylbenzothiazoline-6-sulfonic acid); Sigma-Aldrich] into a stable cationic radical ABTS.⁺ (absorption coefficient, ε=34 220 M⁻¹·cm⁻¹) in tartaric buffer (100 mM) at pH 3. One unit is defined as the amount of enzyme that oxidizes 1 μmol of ABTS per minute. The increase in absorbance at 414 nm was monitored during 1 min with a Carry 50 spectrophotometer at temperature 25° C.

### Reaction Media:

18.9 g of 3-amino-4-hydroxybenzenesulfonic acid as the substrate for producing compound 18 was dissolved in 80 ml of 1 mM tartaric acid (adjusted at pH 5 from strong NaOH) and 100 U of laccase is added. The volume of the mixture was made up to 100 ml by addition appropriate amount of 1 mM tartaric acid (adjusted at pH 5 from strong NaOH). The mixture was subsequently shaken for 24 hours (140 rpm+/−20 rpm) at 24° C.+/−2° C. After 24 hours coloured solution containing compound 18 was filtering off and after freeze drying a brown powder of the compound 18 was obtained.

The product was characterised as follows:

¹H NMR (DMSO, 400 MHz): δ=7.84 (1H, d, J_{H-H}=1.5 Hz, Ar—H), δ=7.65 (1H, dd, J_{H-H}=8.5 and 1.5 Hz, Ar—H), δ=7.44 (1H, d, J_{H-H}=8.5 Hz, Ar—H), δ=6.81 (2H, bs, NH₂), δ=6.38 (1H, s, H-2), δ=6.36 (1H, s, H-5).

¹³C NMR (DMSO, 100 MHz): δ=180.1 (C═O), 176.4 (C═N), 149.0, 145.6, 143.5, 141.5, 132.4, 126.14, 124.2, 115.1.

MW: C₁₂H₈N₂O₅S, 292.3 g/mol.

ES/MS: (m/z) (%)=291 [M-1]⁻

IR (KBr): υ=3441, 3340, 2349, 1600, 1183, 1098, 103

UV: λₘₐₓ at 420-436 nm

Compound 18 has an orange colour. It has favourable staining properties, especially colour fastness, making it highly suitable for colouring textiles, hair and other articles.

### F) Textile Dyeing (SETAS)

The fastness properties of the dyeings (textile and leather) were measured in accordance with ISO standards mentioned in Table 3. The results shown in Table 3 are the visual assessment described in ISO105-A02: 1993 textiles—test for colour fastness—part A02. On this scale, a rating of 5 means no colour change and a rating of 1 means a high colour change.

TABLE 3
Results of experiments in accordance with ISO standards to measure color
fastness of compounds 17 and 18.
Dyeing properties of compound 17 on polyamide 6 knitted fabric

| (2% omf) | | | | | | |
|---|---|---|---|---|---|---|
| Light fastness | | >3 | | | ISO 105 B02 | |
| Washing fastness | | | | | ISO 105 C06: A2S | |
| Colour change | | 3 | | | | |
| Staining | Cellulose | Cotton | Nylon | Polyester | Acrylic | Wool |
| | acetate | 4/5 | 3/4 | 4/5 | 4/5 | 4 |
| | 4/5 | | | | | |
| Water fastness | | | | | ISO 105 E01 | |
| Colour change | | 3 | | | | |
| Staining | Cellulose | Cotton | Nylon | Polyester | Acrylic | Wool |
| | acetate | 4/5 | 3/4 | 4/5 | 4/5 | 4 |
| | 4/5 | | | | | |

| | |
|---|---|
| Chlorinated water | ISO 105 E03 |
| fastness | |

| | |
|---|---|
| Colour change | 3 |

Dyeing properties of compound 18 on polyamide 6 knitted fabric

(1% omf)

| Light fastness | | 3 | | | ISO 105 B02 | |
|---|---|---|---|---|---|---|
| Washing fastness | | | | | ISO 105 C06: A2S | |
| Colour change | | 4 | | | | |
| Staining | Cellulose | Cotton | Nylon | Polyester | Acrylic | Wool |
| | acetate | 4/5 | 4/5 | 4/5 | 4/5 | 4/5 |
| | 4/5 | | | | | |
| Water fastness | | | | | ISO 105 E01 | |
| Colour change | | 4 | | | | |
| Staining | Cellulose | Cotton | Nylon | Polyester | Acrylic | Wool |
| | acetate | 4 | 3 | 4/5 | 4/5 | 2/3 |
| | 4 | | | | | |

| | | |
|---|---|---|
| Chlorinated water | | ISO 105 E03 |
| fastness | | |
| Colour change | 3/4 | |

### G) Toxicity Testing

The toxicity of compounds 17 and 18 were assessed with an in vitro method using the Caco-2 human intestinal cell line and the rainbow trout gonad (RTG-2) fish cell line. Briefly, the cells were seeded in their respective standard cell culture mediums at an appropriate density, and allowed to attach and start proliferation in 96-well microplates in such a way that 20% confluence was reached in 24 hr. Subsequently, the cells were exposed to a series of concentrations of the test compounds, and two blanks, for at least 48 hr until 90% confluence was attained in the blanks. The highest concentration tested being 0.5 g/L. Thereafter, the exposure medium containing the test compounds was decanted and the cells in the microplate wells were gently washed with phosphate buffered saline solution. In order to measure the number of viable cells remaining after the exposure to the test compounds, the cells were exposed to a solution of neutral red in cell culture medium for 3 hr. Viable cells take up the neutral red, while dead cells do not. Finally, the neutral red solution was decanted, the wells were again gently washed and then a solution of 1% acetic acid in 50% ethanol was added to the wells to dissolve the neutral red taken up by the viable cells. The absorbance of each well at 540 nm was measure with an appropriate microplate reader. The amount of neutral red present in each well is an indication of the number of viable cells and when compared to the values for the blanks can be used to calculate a percentage of cell death. An IC₅₀ value, the concentration of the test compounds at which 50% of the cells die, can be determined if at least one of the tested compounds produces more than 50% cell death and at least one produces less; the results concerning compounds 17 and 18 are given in Table 4. If none of the tested concentrations produces more than 50% cell death, than an estimating can be made if at least some effect is noticeable. Comparing IC₅₀ values among test compounds and with known toxicants allows for an assessment of their relative toxicities.

TABLE 4
IC₅₀ values for Compounds 17 and 18

| | Caco-2 cells | NRTG-2 cells |
|---|---|---|
| Compound | IC₅₀ in g/L | IC₅₀ in g/L |
| 17 | >0.55 | >0.78 |
| 18 | >0.686 | |

When expressing IC₅₀ values in g/L, in this particular in vitro method a value of less than 0.01 g/L is attained by highly toxic compounds, a value of less than 0.1 g/L by significantly toxic ones and values above 0.1 g/L by compounds with no significant in vivo toxicity. The compounds shown in the table all have IC₅₀ values which are significantly higher than 0.1 g/L and thus can be considered non-toxic.

What is claimed is:

1. A compound of structural formula (I), a tautomer, a quaternary form or a salt thereof, 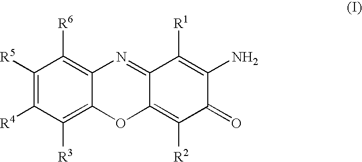
wherein
R¹ is selected from a group consisting of H, —SO₃H, —SO₃M, —SO₂NR⁷R⁸, —SO₂H, —SO₂—C₁₁alkyl, —SO₂—C₆₋₁₂aryl, —SO₂O—C₁₋₆alkyl, and —SO₂O—C₆₋₁₂aryl;
R² is selected from the group consisting of H, —SO₃H, —SO₃M, —SO₂NR⁹R¹⁰, —SO₂H, —SO₂—C₁₋₆alkyl, —SO₂—C₆₋₂₁aryl, —SO₂O—C₁₋₆alkyl, —SO₂O—C₆₋₁₂aryl, C₁₋₆alkyl, C₆₋₁₂aryl, SH, OH, —NH₂, halogen, carboxyl, aminocarbonyl, C₁₋₆alkylaminocarbonyl, and C₆₋₁₂arylaminocarbonyl;
R³ is selected from the group consisting of H, —SO₃H, —SO₃M, —SO₂NR¹¹R¹²—SO₂H, —SO₂—C₁₋₆alkyl, —SO₂—C₆₋₁₂aryl, —SO₂O—C₁₋₆alkyl, —SO₂O—C₆₋₁₂aryl, C₁₋₆alkyl, C₆₋₁₂aryl, SH, OH, —NH₂, halogen, carboxyl, aminocarbonyl, C₁₋₆alkylaminocarbonyl, and C₆₋₁₂arylaminocarbonyl;
R⁴ is selected from the group consisting of H, —SO₃H, —SO₃M, —SO₂NR¹³R¹⁴, —SO₂H, —SO₂—C₁₋₆alkyl, —SO₂—C₆₋₁₂aryl, —SO₂O—C₁₋₆alkyl, —SO₂O—C₆₋₁₂aryl, halogen, aminocarbonyl, C₁₋₆alkylaminocarbonyl, C₆₋₁₂arylaminocarbonyl, C₁₋₆alkyl, C₆₋₁₂aryl, SH, OH, and —NH₂,
R⁵ is selected from the group consisting of H, —SO₃H, —SO₃M, —SO₂NR¹⁵R¹⁶, —SO₂H, —SO₂—C₁₋₆alkyl, —SO₂—C₆₋₁₂aryl, —SO₂O—C₁₋₆alkyl, —SO₂O—C₆₋₁₂aryl, halogen, aminocarbonyl, C₁₋₆alkylaminocarbonyl, C₆₋₁₂arylaminocarbonyl, C₁₋₆alkyl, C₆₋₁₂aryl, SH, OH, and —NH₂;
R⁶ is selected from the group consisting of H, —SO₃H, —SO₃M, —SO₂NR¹⁷R¹⁸, —SO₂H, —SO₂—C₁₋₆alkyl, —SO₂—C₆₋₁₂aryl, —SO₂O—C₁₋₆alkyl, and —SO₂O—C₆₋₁₂aryl;
wherein at least one of R¹, R², R³, R⁴, R⁵ and R⁶ is one of the —SO₂— or —SO₃— containing moieties listed above,
wherein
R⁷ and R⁸ are each independently selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, and C₆₋₁₂aryl;
R⁹ and R¹⁰ are each independently selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, and C₆₋₁₂aryl;
R¹¹ and R¹² are each independently selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, and C₆₋₁₂aryl;
R¹³ and R¹⁴ are each independently selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, and C₆₋₁₂aryl;
R¹⁵ and R¹⁶ are each independently selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, and C₆₋₁₂aryl;
R¹⁷ and R¹⁸ are each independently selected from the group consisting of H, C₁₋₆alkyl, C₁₋₆alkylaminoC₁₋₆alkyl, aminoC₁₋₆alkyl, C₃₋₈cycloalkyl, C₁₋₆alkoxycarbonylC₁₋₆alkyl, hydroxyC₁₋₆alkyl, and C₆₋₁₂aryl, and
M is selected from the group consisting of Li, Na, K, Cs, and Rb.

2. The compound according to claim 1, wherein R² is hydrogen and R¹, R³, R⁴, R⁵, R⁶ have the meaning as defined in claim 1.

3. The compound according to claim 1, wherein R³ is hydrogen and R¹, R², R⁵ and R⁶ have the meaning as defined in claim 1.

4. The compound according to claim 1, wherein R² and R³ are each independently hydrogen and R¹, R⁴, R⁵ and R⁶ have the meaning as defined in claim 1.

5. The compound according to claim 1 having the structural formula (II) 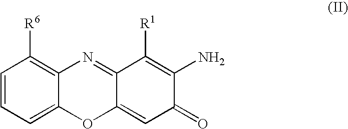
wherein R¹ and R⁶ have the meaning defined in claim 1.

6. The compound according to claim 5 having structural formula (II), wherein R¹ is H, and R⁶ is has the meaning defined in claim 1.

7. The compound according to claim 5, having structural formula (II) wherein R¹ is has the meaning defined in claim 1, and R⁶ is H.

8. The compound according to claim 5 having structural formula (II), wherein
R¹ is selected from a group consisting of —SO₃H, —SO₃M, —SO₂NR⁷R⁸, —SO₂—C₁₋₆alkyl, —SO₂—C₆₋₁₂aryl, —SO₂O—C₁₋₆alkyl, and —SO₂O—C₆₋₁₂aryl,
R⁶ is selected from the group consisting of —SO₃H, —SO₃M, —SO₂NR¹⁷R¹⁸, —SO₂—C₁₋₆alkyl, —SO₂—C₆₋₁₂aryl, —SO₂O—C₁₋₆alkyl, and —SO₂O—C₆₋₁₂aryl, and
R¹⁷ and R¹³ have the meaning as defined in claim 1.

9. The compound according to claim 1 having the structural formula (III) 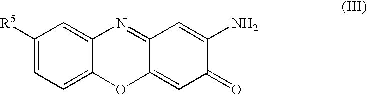
wherein R⁵ has the meaning defined in claim 1.

10. The compound according to claim 9 having the structural formula (III), wherein
R⁵ is selected from a group consisting of —SO₃H, —SO₃M, —SO₂NR¹⁵R¹⁶, —SO₂—C₁₋₆alkyl, —SO₂—C₆₋₁₂aryl, —SO₂O—C₁₋₆alkyl, and —SO₂O—C₆₋₁₂aryl, and
R¹⁵ and R¹⁶ have the meaning as defined in claim 1.

11. The compound according to claim 1 that is a compound selected from the group consisting of 2-amino-3-Oxo-3H-phenoxazine-1,9-disulfonic acid diamide, 2-amino-3-oxo-3H-phenoxazine-1,9-disulfonic acid bis-phenylamide, 2-amino-3-oxo-3H-phenoxazine-1,9-disulfonic acid bis-cyclohexylamide, 2-amino-3-oxo-3H-phenoxazine-1,9-disulfonic acid bis-[(3-dimethylamino-propyl)-amide, 2-amino-3-oxo-3H-phenoxazine-1,9-disulfonic acid bis-[(2-amino-ethyl)-amide], 2-amino-[9-(methoxycarbonylmethyl-sulfamoyl)-7-oxo-7H-phenoxazine-1-sulfonylamino]-acetic acid methyl ester, 2-amino-3-oxo-3H-phenoxazine-1,9-disulfonic acid bis-[(3-hydroxypropyl)-amide].

12. A method for the preparation of a compound of formula (I) as defined in claim 1, comprising the step of catalytically dimerising an aromatic precursor having structural formula (IV), 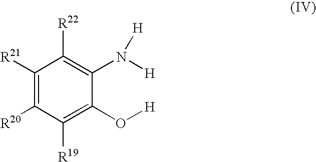
where R¹⁹ is the same as R² or R³ as defined in claim 1, R²⁰ is the same as R⁴ as defined in claim 1, R²¹ is the same as R⁵ as defined in claim 1, and R²² is the same as R¹ or R⁶ as defined in claim 1, whereby the catalysis proceeds using a laccase enzyme.

13. Method according to claim 12, wherein the laccase enzyme is in free solution or immobilised on a solid support, or is provided by a micro-organism containing a gene for laccase enzyme expression.

14. Method according to claim 12, wherein the reaction proceeds in an aqueous medium.

15. Method according to claim 12, wherein the medium further comprises an alcohol at a concentration of between 1 and 10% (v/v).

16. Method according to claim 12, wherein the laccase enzyme is present at a concentration that provides an activity of between 1 and 1000 U·L⁻¹.

17. Method according to claim 12, wherein the compound of formula (IV) is present at a concentration of between 1 and 10 g·L⁻¹.

18. Method of dyeing an article comprising the step of contacting said article with a compound according to claim 1.

19. An article dyed using a compound as defined in claim 1.
